(19) United States
(12) Patent Application Publication
(10) Pub. No.: US 2009/0299388 A1
(43) Pub. Date: Dec. 3, 2009

(54) Reinforcement Device

(75) Inventors: Stephen George Edward Barker, Surrey (GB); Graham Emery Steer, London (GB)

Correspondence Address: HARNESS, DICKEY & PIERCE, P.L.C., P.O. BOX 828, BLOOMFIELD HILLS, MI, 48303, US

(73) Assignee: EVEXAR MEDICAL LIMITED, Kent (GB)

(21) Appl. No.: 11/992,214

(22) PCT Filed: Sep. 14, 2006
(86) PCT No.: PCT/GB2006/003410

§ 371 Date: Jul. 28, 2009

(30) Foreign Application Priority Data
Sep. 19, 2005 (GB) 0519095.4

(51) Int. Cl. A61B 17/12; B29C 51/12

(52) U.S. Cl. 606/155; 156/245

(57) ABSTRACT

A device (10) is provided for use in reinforcing a boundary wall of a patient's abdominal cavity at a site at which a portion of the patient's alimentary tract passes through that boundary wall. The device comprises a collar portion (11) for extending, in use, around the alimentary tract (17) at the approach of the alimentary tract to the interior of the boundary wall at said site. The device also comprises a plurality of petal-like elements (12) extending from the collar portion (11), said elements being arranged so as to extend, in use, outwardly with respect to the longitudinal axis of the portion of the alimentary tract passing through the collar portion and to be attached, in use, to the interior of the boundary wall around said side. The device is preferably, but not exclusively, for use in the prevention or treatment of parastomal herniation in patients having a colostomy or ileostomy or the like. It may however also find other applications, for example in the repair of developed hiatus hernias or the like.

This invention relates to a device for use in reinforcing a boundary wall of a patient's abdominal cavity at a site at which a portion of the patient's alimentary tract passes through that boundary wall. The device is preferably, but not exclusively, for use in the prevention or treatment of parastomal herniation in patients having a colostomy or ileostomy or the like. It may however also find other applications, for example in the repair of developed hiatus hernias or the like.

A stoma is an artificial opening of the intestine on to the abdominal wall. Less commonly, it is an artificial opening from the urinary tract, called a urostomy. Parastomal herniation is where a hernia forms adjacent the site of the stoma.

A stoma is necessary when bowel surgery has removed important other lengths of the intestine, for example with cancer or inflammatory bowel disease. The main different types of stoma depend on the part of the intestine that forms the opening. An ileostomy is an opening from the small bowel (ileum) to allow very liquid faeces to leave the body without passing through the large bowel. A colostomy is an opening from the large bowel to allow faeces to bypass the rectum or anal canal.

Other procedures involving the formation of a stoma include: gastrostomy, jejunostomy and urostomy.

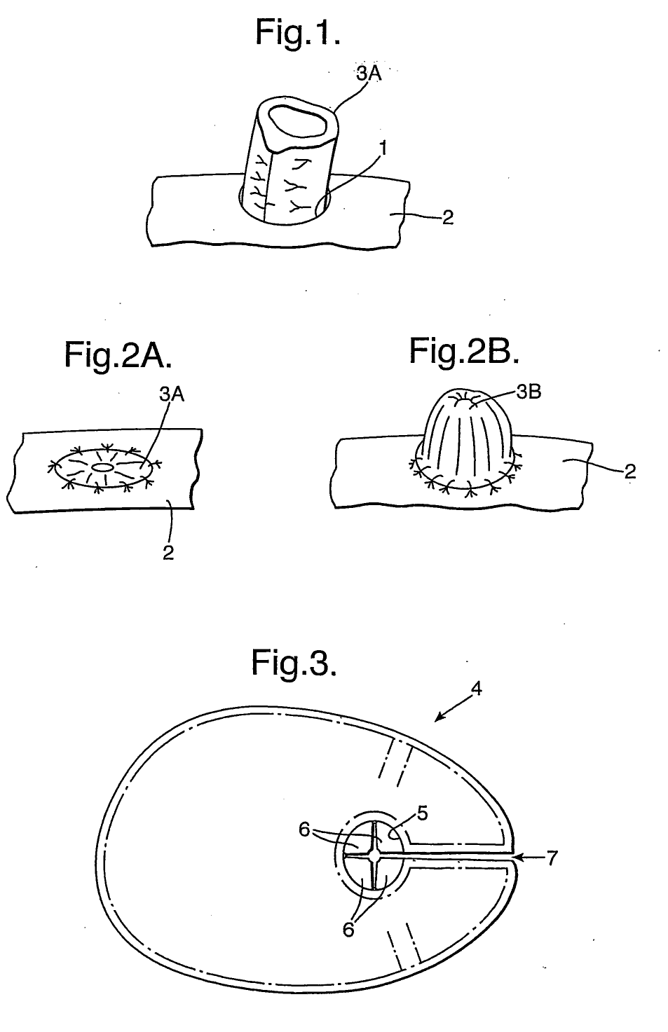

The general procedure for formation of a stoma, such as for example with a colostomy or ileostomy, is illustrated in FIGS. 1 and 2A and 2B. In FIG. 1 an opening 1 is shown as having been formed in the abdominal wall 2. The end of the intestine 3A is then pulled through the opening 1. The end of the intestine 3A may then be sutured ‘flush’ to the skin for a colostomy for example, as shown in FIG. 2A. Alternatively it may be folded outwards, back onto itself, and attached to the abdominal wall 2 using a series of stitches around the perimeter of the opening 1, thereby to form the stoma 3B, as a ‘spouted’ ileostomy for example, as shown in FIG. 2B.

Conventionally, the abdominal wall around the internal abdominal opening is not ‘reinforced’ during the formation of an ileostomy, or colostomy. It is estimated that 30% to 50% of all colostomies and ileostomies result subsequently, in parastomal herniation (Ref: British Journal of Surgery, Volume 90, Issue 7, July 2003) as a result of the created opening in the abdominal wall weakening the abdominal wall. If the weakened abdominal wall in the region of the stoma is stretched over time, a parastomal hernia can form allowing the underlying intestines' to bulge through the abdominal wall adjacent the stoma.

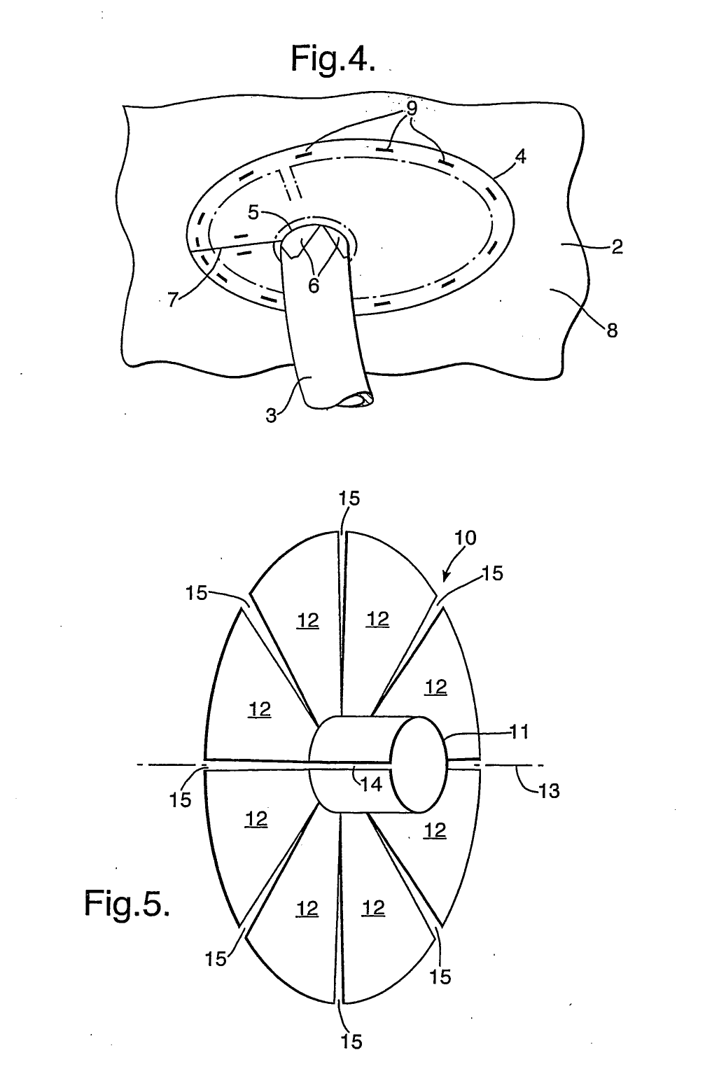

At present the formation of a parastomal hernia results in the patient having to undergo a subsequent major surgical procedure. In an extreme case, this might involve relocation of the stoma to another location on the abdominal wall which is as highly skilled, expensive and highly traumatising procedure. In less extreme cases the abdominal wall around the site of the stoma may be reinforced using a parastomal hernia patch. One such patch is sold by Davol Inc., a subsidiary of C. R. Bard, Inc., as the BARD CK (Trademark) Parastomal Hernia Patch This patch is not intended primarily to be used as a prophylactic device, to prevent parastomal herniation. It is, instead, primarily for the patch repair of developed parastomal hernias. As shown in FIG. 3, the patch 4 comprises a generally oval, flat sheet of material made up of a sheet of expanded polytetrafluoroethylene (ePTFE) attached, via stitching shown as broken lines, to a correspondingly shaped sheet of polypropylene mesh A central, circular region 5 of the patch comprises only the ePTFE material and not the polypropylene mesh, and is formed into four floppy “wings” 6. This region 5 is to receive the intestine 3. A radial slit 7 is provided between the extremity of the patch and the circular region 5 so as to enable the patch to be positioned around the site of the stoma, with the largest portion of the patch covering the parastomal hernia defect, and with the ePTFE surface forming the visceral surface and the polypropylene mesh surface in contact with the abdominal wall (around the site of the stoma). In its final position (shown in FIG. 4) the four floppy “wings” 6 are loosely folded up the side of the intestine immediately prior to the point at which the intestine passes through the central region 5 of the patch 4. The patch 4 is fixed to the interior of the abdominal wall 8, for example using surgical staples 9. The patch is provided with a couple of generally C-shaped “memory recoil rings”, around the outer periphery of the patch and around the periphery of the central region 5, to assist the patch to spring open and maintain its shape at the repair site. These rings, however, contribute to the patch wanting to lie flat against the (usually curved) interior abdominal wall. In addition, provision of only a single radial slit 7 in the patch 4, and the flat nature of the patch, can present the surgeon with difficulties in conforming the patch to the curved interior surface of the abdominal wall 2 around the site of the stoma and the hernia defect. Good conformity and contact between the non-visceral surface of the patch and the interior wall of the abdomen are helpful in promoting tissue ingrowth from the interior of the abdominal wall into the polypropylene mesh, and help to increase the level of reinforcement provided by the patch.

There is a need for a primarily prophylactic device for use in reinforcing a colostomy or ileostomy site, or the like, to prevent the formation of a parastomal hernia in the first place, avoiding the need for a post-colostomy or post-ileostomy surgical procedure to repair a parastomal hernia. Furthermore, there is a need for the device to be readily conformable to a patient's abdominal wall around the site of a stoma so as to maximise the level of incorporation into the abdominal wall and hence the reinforcement offered by the device. By placing the device around the intestine, inside the abdominal cavity, at the time of the colostomy or ileostomy surgical procedure, herniation risk should be substantially reduced or even eliminated.

According to a first aspect of the present invention there is provided a device for use in reinforcing a boundary wall of a patient's abdominal cavity at a site at which a portion of the patient's alimentary tract passes through that boundary wall, the device comprising:
a collar portion for extending, in use, around the alimentary tract at the approach of the alimentary tract to the interior of the boundary wall at said site; and
a plurality of petal-like elements extending from the collar portion, said elements being arranged so as to extend, in use, outwardly with respect to the longitudinal axis of the portion of the alimentary tract passing through the collar portion and to be attached, in use, to the interior of the boundary wall around said site.

Where the device is for use in the prevention or treatment of parastomal herniation in patients having a colostomy or ileostomy or the like, the boundary wall is the patient's abdominal wall, the portion of the alimentary tract is the patient's intestine and the site is the site of the stoma.

Although the device is primarily intended for use as a prophylactic device to limit or reduce known complications, thus saving significant financial cost, later complications and patient trauma, it is also intended that the device can be used in revisional surgery to effect a repair to a developed parastomal hernia at the site of a previous colostomy or ileostomy procedure.

Where, however, the device is for use in the repair of a developed hiatus hernia, the boundary wall is the patient's diaphragm and the portion of the alimentary tract is the patient's oesophagus at the point at which it passes through the diaphragm.

According to a second aspect of the present invention there is provided a method of reinforcing a boundary wall at a patient's abdominal cavity at a site at which a portion of the patient's alimentary tract passes through that boundary wall, the method comprising:
providing the device of the above first aspect of the present invention;
forming the collar portion around the alimentary tract at the approach of the alimentary tract to the interior of the boundary wall at said site, with said plurality of petal-like elements in contact with the interior of the boundary wall around said site; and
fastening said plurality of petal-like elements to the contacting interior boundary wall around said site.

According to a third aspect of the present invention there is provided a method of manufacturing the device of the above first aspect of the present invention, the method comprising:
a) providing a sheet of biocompatible plastics material; and
b) forming said sheet into the collar portion and the plurality of petal-like elements by molding and cutting.

Embodiments of device in accordance with the present invention will now be described, by way of example only, with reference to the accompanying drawings, in which:

FIG. 1 illustrates a first stage in a typical procedure involving the formation of a stoma;

FIGS. 2A and 2B illustrate typical subsequent stages in the formation of flush and spouted stomas respectively;

FIG. 3 illustrates a prior art parastomal hernia patch similar to the BARD CK (trademark) patch in top plan view;

FIG. 4 illustrates the patch of FIG. 3 in situ in a typical parastomal hernia repair situation, pictured from inside the patient's abdomen, with the patient's intestines (save for the portion of the intestines extending through the patch and abdominal wall to form the stoma) being omitted for reasons of clarity, with the patch shown fixed to a portion of abdominal wall interior;

FIG. 5 is a perspective view of a first embodiment of device in accordance with the present invention, shown prior to use;

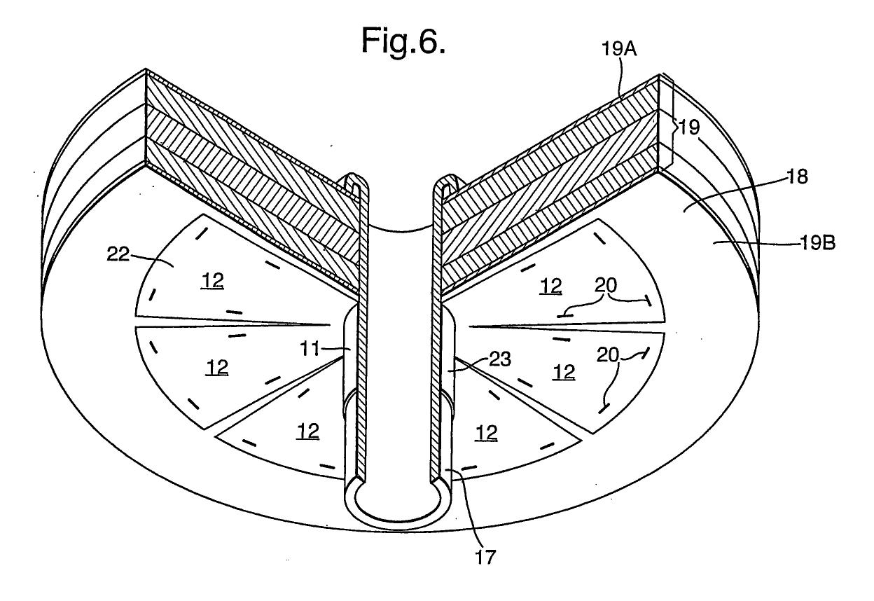

FIG. 6 is a perspective view of the FIG. 5 embodiment device in use and from below, viewed from inside the patient's abdominal cavity, with a quarter section of the abdominal wall, the device and the intestine cut away and showing the device's petal-like elements contacting the internal surface of the abdominal wall with the intestine snugly received within the device's collar portion;

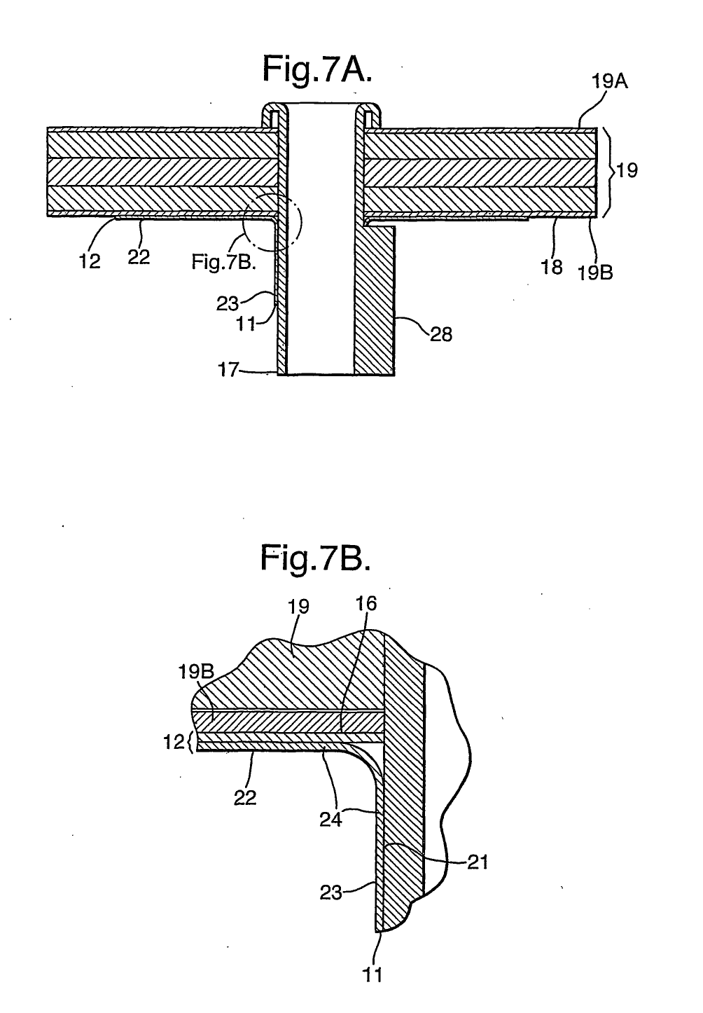

FIG. 7A is a sectional side elevation of the FIG. 5 embodiment of the device in use, showing the device attached to the interior of a patient's abdominal wall with the mesentry extending through the opening provided in the device's collar portion;

FIG. 7B is an enlarged, close-up view of the portion of the device ringed in FIG. 7A, showing two layers of material making up the device's generally planar elements, with a single layer of material (continuous with the visceral layer of the generally planar element) extending through approximately 90° to form the device's collar portion;

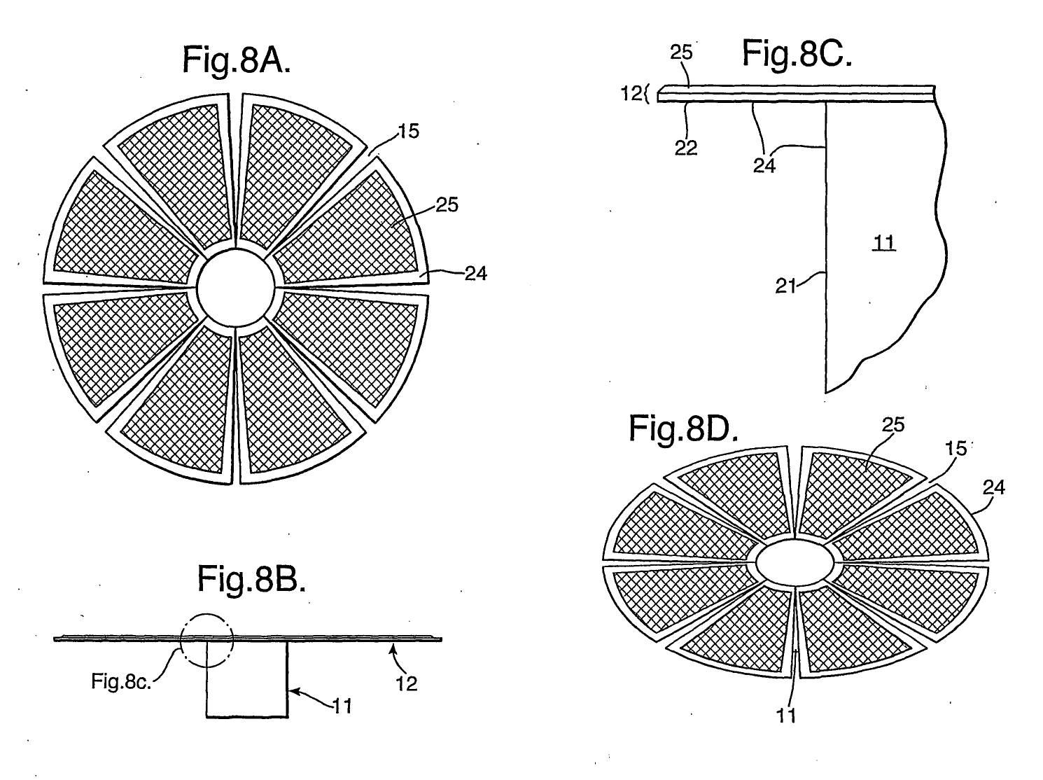

FIG. 8A is a top-plan view of the FIG. 5 embodiment of device;

FIG. 8B is a side elevation of the FIG. 5 embodiment of device.

FIG. 8C is an enlarged close-up of the portion of the device ringed in FIG. 8B; 15

FIG. 8D is a perspective view of the FIG. 5 embodiment of device, from above and to one side;

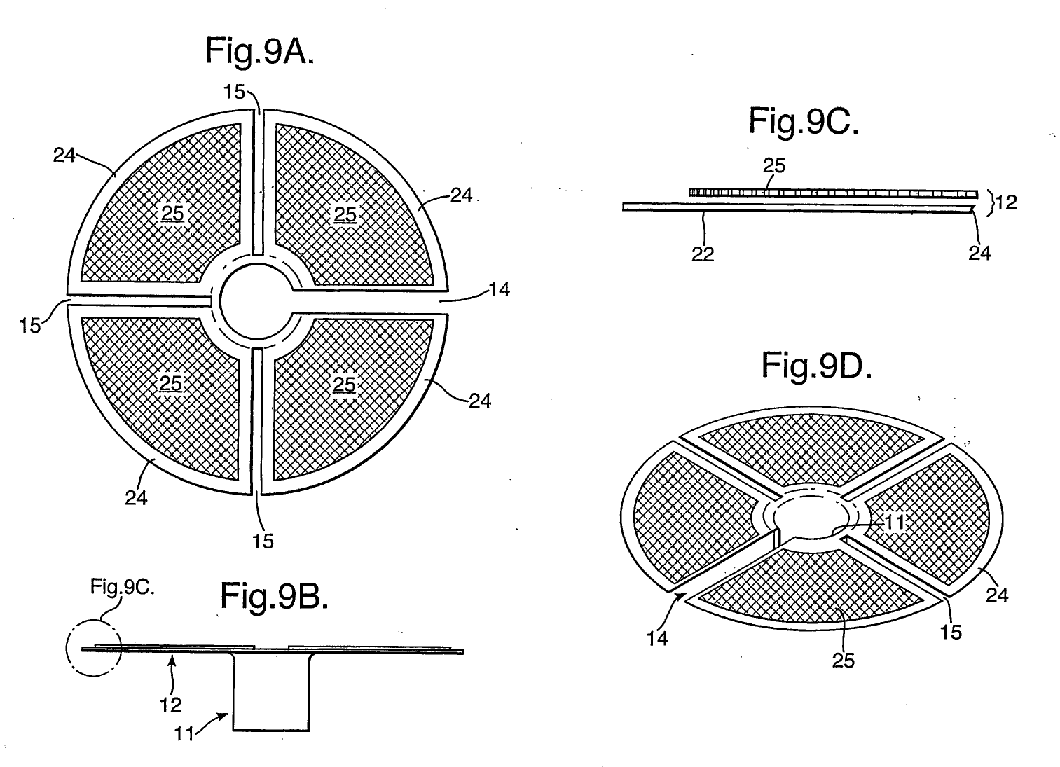

FIG. 9A is a top plan view of a second embodiment of device, having fewer petal-like elements and a larger radial gap than the first embodiment of device;

FIG. 9B is a side elevation of the second embodiment of device;

FIG. 9C is an enlarged close-up view of the section of the device ringed in FIG. 9B, but showing the two layers of material of the generally planar element spaced apart to improve clarity;

FIG. 9D is a perspective view of the second embodiment of device from above and to one side;

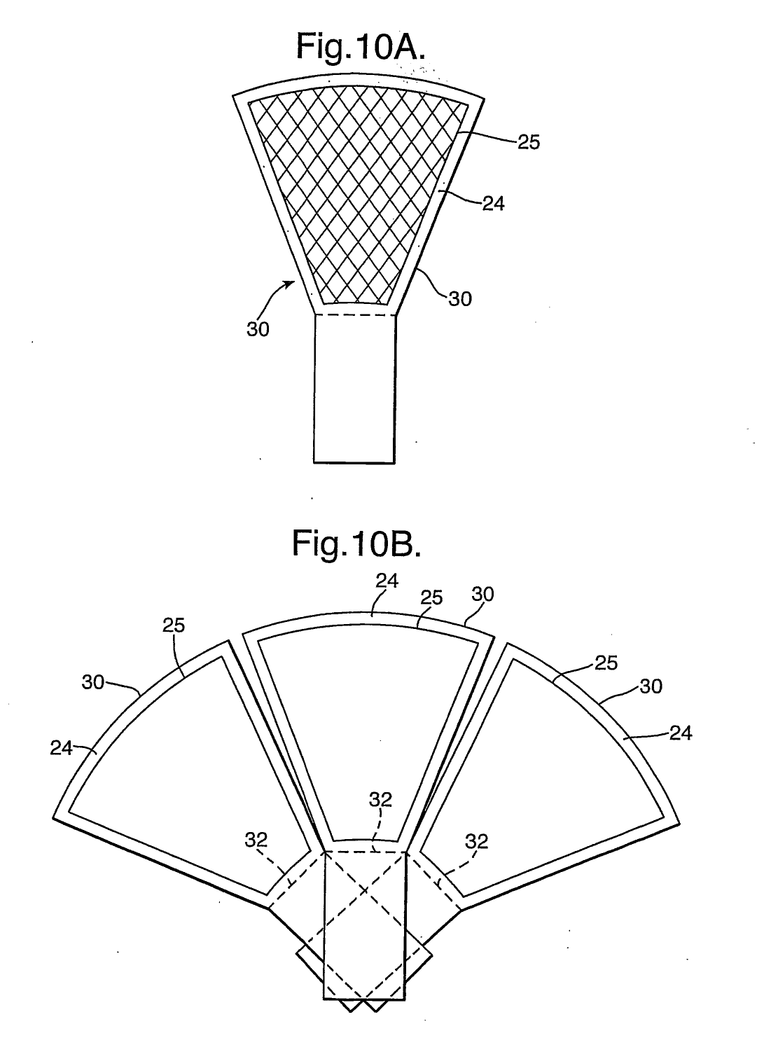

FIG. 10A is a top plan view of a first element of material for use in the manufacture of the first embodiment of device;

FIG. 10B is a top plan view showing three of the FIG. 10A elements arranged together during the manufacturing process; and

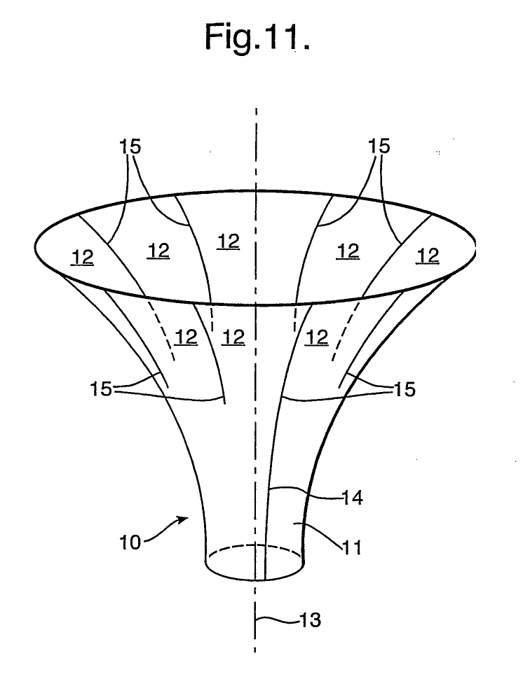

FIG. 11 is a perspective view of a third embodiment of device in accordance with the present invention, shown prior to use.

A first embodiment of device 10, ideally a parastomal hernia prophylactic device, is illustrated in FIGS. 5-8. As will become apparent, one use of the device is in reinforcing a site at which a patient's intestine is brought through to the patient's abdominal wall to form a stoma, for example in a colostomy or ileostomy formation procedure. This is the procedure that will be described below, by way of example. As mentioned earlier, when used in conjunction with a colostomy or ileostomy the device is primarily intended to be used as a prophylactic device, to be implanted during the primary colostomy or ileostomy procedure so as to eliminate (or at least significantly reduce) the risk of subsequent parastomal herniation. The device 10 may, however, be used in a subsequent revisional surgery procedure to treat an existing hernia defect, such as parastomal hernia or a hiatus hernia.

Although the majority of the following description will describe the use of the device as a prophylactic parastomal hernia device, this description is by way of example only and is non-limiting. For the sake of simplicity, the other potential clinical uses of the device (mentioned elsewhere in the description) will not continuously be referred to. For example, where the terms abdominal wall and intestine are used, these could equally refer instead to diaphragm and oesophagus if the device was being used to treat a developed hiatus hernia.

At least immediately prior to use, and as shown in FIG. 5, the device 10 comprises a generally tubular collar portion 11 for extending, in use, around the intestine at the approach of the intestine to the interior of the abdominal wall at the site of the stoma. The device further comprises a plurality (eight in the illustrated embodiment) of generally planar, petal-like elements 12 extending from one end of the collar portion 11 generally radially with respect to the longitudinal axis 13 of the tube of the collar portion 11. As will become apparent, in addition to extending generally radially with respect to the longitudinal axis 13, the elements 12 will in use, extend generally radially outwardly with respect to the longitudinal axis of the portion of intestine (not shown in FIG. 5) passing through the collar portion 11, because the two longitudinal axes will in use, be generally co-axial. By extending “generally radically outwardly” is meant extends with a significant radial component—the term does not simply mean extending purely or nearly purely radially.

As will also become apparent later, the elements 12, in use, are flexible and independently manipulatable so as to enable them to conform readily to the shape of the interior of the abdominal wall around the site of the stoma and to be attached thereto using conventional known fastening means, such as staples or sutures.

Although the collar portion 11 is generally tubular, it will be noted that it is provided with a radial gap 14 along its full axial length, representing a sector that is “missing” from the otherwise generally rotationally symmetrical device. This gap 14 is so as to enable the device to be fitted around the intestine in use, by passing the intestine through the gap 14, as well as to provide a gap in the collar portion 11 for the intestine's mesentry 28 to extend through, for the continued supply of blood (via the mesentry) to the portion of the intestine that is received in use within the collar portion 11, as shown in FIG. 7A.

As will be described in more detail below, the individual elements 12 are separated from one another by generally radially directed slits 15. FIG. 5 represents the likely configuration of the device immediately prior to its implantation. The device 10 may, however, alternatively be provided to the surgeon in a different configuration, for example, a partially or completely flattened configuration more compatible with compact packaging of the device in a sterile peelable thermoformed tray, requiring the surgeon or a surgery assistant to manipulate the device into the final configuration shown in FIG. 5 prior to its implantation.

An exemplary procedure for the implantation of the device of FIG. 5 will now be described, by way of example, as part of a colostomy or ileostomy procedure. The device is implanted after the formation of the stoma, the site of which the device 10 is to reinforce. Implantation may be during the same surgical procedure as the formation of the stoma (where the device is intended to be a prophylactic device), or 5 may be during a subsequent revisional surgical procedure so as to repair an existing parastomal hernia defect. The procedure will be described in conjunction with use of the device as a prophylactic device, but the procedure for use in revisional surgery would be generally similar, save that the surgeon would need to ensure that the elements 12 were positioned overlapping the hernia defect and extending some distance (for example 3-5 cm) beyond the defect.

With access to the portion of the intestine at the approach of the intestine to the interior of the abdominal wall at the site of the stoma, the surgeon first needs to select an appropriately sized device and then to orient the device 10 correctly. The correct orientation of the device 10 is with the end of the collar portion 11 that is provided with the elements 12 positioned closest to the interior of the abdominal wall. With the device 10 in this orientation, the device is positioned around that portion of the intestine by passing that portion of the intestine radially through the gap 14 in the collar portion 11. Once this is done, the collar portion 11 of the device 10 (which is flexible) can be closed around that portion of the intestine to bring the generally cylindrically inner (non-visceral) wall of the collar portion 11 into snug contact with the exterior of the intestine portion. As shown in FIG. 7A, the mesentry 28 of that received intestine portion, which is now received in the collar portion 11, is aligned with the gap 14 in the collar portion 11 so that it can extend through the gap 14 and need not be cut away internally of the interior abdominal wall, and can thereby continue to supply nutrition to the received portion of the intestine. If necessary, the gap 14 can be enlarged by the surgeon, by cutting away a portion of the material of the collar portion 11. As shown in FIGS. 6, 7A and 7B, in this final position of the device 10 the non-visceral surfaces 16 of the elements 12 are in contact with the interior surface 18 of the abdominal wall 19 around the site of the stoma and, due to the flexible nature of the elements 12, can conform precisely to the interior (often generally concave) shape of the interior surface of the abdominal wall. In the drawings, the abdominal wall 19 is shown as made up of three main layers sandwiched between a thin external layer 19A (the epidermis) and a thin internal layer 19B (the peritoneum).

It will be appreciated that the provision of the slits 15 between the elements 12 enables the elements 12 to deflect independently of one another and to conform more readily to the interior shape of the abdominal wall 19 without substantial creasing, in contrast to the situation if the slits 15 were not present and the elements were instead one continuous planar C-shaped sheet. By improving the matching between the elements 12 and the interior shape of the abdominal wall 19, the area of contact between the non-visceral surfaces 16 of the elements 12 and the interior surface 18 of the abdominal wall 19 can be increased, thereby increasing the potential for the ingrowth of tissue from the abdominal wall 19 into the elements 12, as described 15 below. Increasing the potential for incorporation of the elements 12 into the abdominal wall 19 in this way improves the level of reinforcement to the abdominal wall 19 provided by the device 10.

With the device 10 in its final position, the surgeon can fix the elements 12 to the interior of the abdominal wall around the site of the stoma using known fastening means and techniques (for example staples, tacks or sutures 20—see FIG. 6).

Although not thought to be essential, the collar portion 11 may be attached to the received intestine portion 17, for example using non-absorbable sutures (not shown). This may have benefits in improving the level of guidance and direction of the intestine to the skin surface 19A provided by the device 10, as well as to reduce the risk of herniation through intussusception.

FIG. 7A shows the intestine's mesentry 28 extending through the gap 14 (not shown) in the collar portion 11 of the device 10. To provide support to the received portion of the intestine 17 the internal (non-visceral) surface 21 of the collar portion 11 and the external surface of the received portion of intestine 17 are in snug contact with one another. FIG. 7A, and especially FIG. 7B, also shows the (non-visceral) surfaces 16 of the elements 12 that face the interior surface 18 of the abdominal wall 19 being in contact with that interior surface 18. FIG. 7A also shows how the received and stoma-forming portion of the intestine 17 is left provided with its mesentry 28 up to the interior surface 18 of the abdominal wall 19. The mesentry 28 need not extend into the abdominal wall 19, because the portion of the intestine received within the opening in the abdominal wall 19 can take its blood supply from the abdominal wall layers.

The elements 12, and collar portion 11, both have a visceral surface (22 and 23 respectively) that will face the patient's internal organs or viscera in use of the device. Adhesion between the internal organs in the body cavity and the device 10 is highly undesirable. Consequently, it is advantageous for the visceral surfaces 22, 23 of the elements 12 and collar portion 11 to comprise, or at least be faced with, a material that will minimise undesirable tissue adhesion. In a preferred arrangement this material 24 is a sheet of polytetrafluoroethylene or expanded polytetrafluoroethylene material. The material 24 may, however, be any other proprietary biocompatible material that is deemed to be “non-stick”.

The elements 12 each have a non-visceral surface 16, facing in the opposite direction to the elements' visceral surfaces 22. These non-visceral surfaces 16 of the elements 12 contact the interior surface 18 of the abdominal wall 19 in use, as shown in FIGS. 7A and 7B. In order to maximise the level of reinforcement offered by the device 10 to the abdominal wall 19 in the vicinity of the stoma, it is highly desirable for there to be tissue ingrowth from the abdominal wall into the non-visceral surface 16 of the elements 12. In this way, the elements 12 become incorporated into the abdominal wall 19 so as to provide reinforcement to the abdomen wall.

In order to encourage tissue ingrowth into the non-visceral surfaces 16 of the elements 12, those surfaces comprise, or at least are faced with, a material 25 which will promote tissue ingrowth. In a preferred arrangement, the non-visceral surfaces 16 of the elements 12 comprise an exposed polypropylene mesh-like material 25. This mesh-like material 25 is represented by a criss-cross weave effect in FIGS. 8 and 9. The mesh-like material 25 may be bare exposed polypropylene mesh, but might alternatively comprise a sheet of polypropylene mesh embedded with collagen or gelatin or the like to encourage adherence of the device to the abdominal during surgery due to stickiness, as well as to act as a haemostat.

To provide the visceral 22 and non-visceral 16 surfaces of the elements 12 with their different properties, each of the elements 12 is preferably, as illustrated, composed of two sheets of material 24, 25. As can be seen from FIGS. 8A and 8D, the mesh-like material (shown as a “crisscross weave”) 25 is overlaid onto the non-stick material 24, with the non-stick material extending beyond the boundary of the mesh-like material. The two materials 24, 25 are advantageously fixed together by stitching or gluing or the like. By extending beyond the periphery of the mesh-like material 25, the non-stick material 24 shields the cut edges of the mesh-like material from inadvertent contact with the viscera, which contact could promote undesirable fistulation.

It is thought that the non-visceral (interior) surface 21 of the collar 11 should not promote the ingrowth of tissue from the received portion of the intestine 17. It is thought to be better for tissue ingrowth from the intestine 17 into the collar portion 11 to be discouraged, because this could lead to undesirable fistulation. Furthermore, if at any time the device 10 needed to be removed and replaced, ingrowth of tissue from the received portion of the intestine 17 into the non-visceral surface 21 of the collar 11 would complicate removal of the device.

For this reason, the non-visceral (interior) surface 21 of the collar portion 11 of the device advantageously has the same non-stick properties as the visceral (exterior) surface 23 of the collar portion. As shown in FIG. 7B, this can be achieved by having a single thickness of the non-stick material, with its opposite surfaces forming both the non-visceral and visceral surfaces 21, 23.

As can be seen from FIG. 5, prior to use the device generally resembles an open-topped top hat, albeit with a large brim that is provided with a plurality (eight as drawn) of radially directed slits 15, with a single radially directed slit 14, 15 extending through the complete “hat”.

To form the device 10 into this 3-D shape, the generally non-stick material 24 can be moulded into this desired form. Alternatively, the device 10 can be built up from a plurality of components. One such technique will now be described, by way of reference to FIGS. 10A.

Firstly, a plurality of Y-shaped elements 30 of the non-stick material 24 maybe formed, for example, by being stamped or cut out of a larger sheet of non-stick material, see FIG. 10A. A portion of mesh-like material 25 may then be fixed, for example by gluing or stitching, to the divergent end of each of the elements 30. The Criss-cross weave of material 25 is shown in FIG. 10A but not FIG. 10B. These divergent ends will, in use, form the generally planar elements 12 of the device 10. The elements 30 may then be arranged in an array (as shown in FIG. 10B) and fixed together, for example by gluing or stitching. In FIG. 10B only three such elements 30 are shown, but as many elements 30 would be joined together as there are to be elements 12 in the finished device 10.

The joined together elements 30 may, in use, be used to form the collar portion 11 of the device. Alternatively, or additionally, a patch of non-stick material maybe sewn onto the stitched together stems. As a yet further alternative, the majority of the overlapped portions of the elements 30 could be cut away prior to stitching on the patch of non-stick material, so that the majority of the device's collar portion 11 is of constant, single-sheet thickness. The further patch of material may be a rectangular patch which is, or can be, formed into a slit cylindrical shape to form the complete collar portion 11.

In order to form the generally top-hat shape of the device, by bending each of the elements over, through approximately a right angle, along line 32 and then forming the device around axis 13 to form the generally cylindrical collar portion 11, the device may be changed from the generally 2-D structure illustrated in FIG. 10B to the generally top hat shaped 3-D structure illustrated in FIG. 5. In order to assist in the manipulation of the device 10, heat may be applied in a known fashion, whilst the device is being manipulated into its final top-hat shape.

Other suitable biocompatible materials for the elements 12 and collar portion 11 of the device will be apparent. For example, the biocompatible material for promoting tissue ingrowth might be a different structural non-absorbable material selected from the group consisting of polypropylene, Dacron, silicon, polyethylene, polyamide, titanium, stainless steel, polymethymethacrylate or polyurethane. An alternative to PTFE or ePTFE for the non-stick material could be any other proprietary biocompatible material deemed to be non-stick.

In use, triangular areas of the interior of the patient's abdominal wall will be left exposed between the opposed lateral edges of adjacent elements 12, as is shown most clearly in FIG. 6. In order to enhance the reinforcement offered by the device 10, it may be desirable to reduce or eliminate the area of the patient's abdominal wall interior that will be left exposed between the elements 12 in use. This can be achieved by making the elements 12 larger than is shown, for example to abut, or even overlap, in use, so that when the elements 12 are fastened to the interior surface 18 of the abdominal wall 19 they will form a substantially continuous O- or C-shaped reinforcing element. In this situation, overlapping would enable the fastenings 20, such as sutures, tacks or staples, to penetrate through the zones of overlap.

As mentioned earlier, the provision of the slits 15 between the individual elements 12 enables the elements 12 more readily to conform to irregular internal shapes for the interior surface 18 of the patient's abdominal wall 19. For example, if the interior of the patients abdominal wall is generally concave (when viewed from the interior of the abdominal wall), the individual flexible elements 12 can readily be conformed to follow the shape of the abdominal wall, and then fastened to the abdominal wall. By increasing the area of contact between the non-visceral surfaces of the elements 12 and the abdominal wall, tissue ingrowth into the elements 12 can be enhanced with a resultant increase in the level of reinforcement provided by the device 10. In addition, the provision of a plurality of individual elements 12 enhances the device's ability to be able to form a snug fit between the intestine 17 and the non-visceral surfaces 21 of the collar portion 11 to enable the collar portion to be attached along its length to the received portion of the intestine to help to prevent intussusception.

It is envisaged that the device will be provided in a range of sizes, to accommodate intestines of different girths. Furthermore, the radial extent of the elements 12 may vary between devices. For example, a device for use in prophylaxis may have a smaller radial extent to the elements 12 than a device for use in revisional surgery, due to the need for the elements 12 to extend over and beyond the parastomal hernia defect in the case of revisional surgery. At the start of a surgical procedure it is envisaged that the surgeon would select from a range of differently dimensioned devices a device that best suits the geometry of the patient's physiology. In addition, the elements 12 may not all have the same radial extent Particularly for use in revisional surgery the elements 12 that will cover the defect may be larger, for example to give the combined elements a more oval appearance than circular.

Although in the Figures the collar portion 11 is shown as being generally tubular and of significant elongate extent, it need not be of such a long length. For example, if the collar portion 11 is not required to provide substantial support to the intestine 17 immediately prior to the intestine's entry into the opening provided in the abdominal wall 19, the collar portion may be no more than a C-shaped element of minimal axial extent, to serve as a means for holding the elements 12 together in an array and allowing them to be drawn radially into contact with the intestine and supported at their radially innermost ends.

Although in the first embodiment of device 10, illustrated in FIGS. 5-8, the device is shown as being provided with eight elements 12, other numbers of elements may be provided. For example, as shown in FIGS. 9A-9D the number of those elements 12 may be reduced and the size of the individual elements increased. FIGS. 9A and 9D also show how the gap 14 provided in the device 10 may be rather larger than in the FIG. 5-8 embodiment.

The device need not be built up from a plurality of components as described in conjunction with FIGS. 10A and 10B, nor need the device have the elements 12 in the form of a flat planar sheet extending from the collar portion 11 at a sharp right-angle. In a third embodiment of device 10 illustrated in FIG. 11 it can be seen how the device may, when freestanding prior to use, generally resemble the cone end of a trumpet. A plurality of slits 15 provided in the bell of the trumpet separate the eight petal-like elements 12 from one another. The obscured lower portions of the back set of slits 15 are shown in dotted lines. A radial gap 14 is provided down the full length of the device for the same reason as the gap 14 in the earlier embodiments of device. As will be appreciated, in contrast to the first and second embodiments of device, in the third embodiment of device 10 there is a radiussed, gradual transition between the collar portion 11 and the petal-like elements 12. Although the distal portions of the elements 12 which are furthest from the collar portion are generally perpendicular to the longitudinal axis of the collar portion, it will be appreciated that the proximal portions of the elements 12 (closer to the collar portion) are less so.

One advantage of the device 10 generally resembling the cone end of a trumpet is that it is particularly suitable for the formation of the device from a single sheet of material. By taking a single sheet of material and molding it, for example by thermo-forming it using the application of both pressure and heat the generally flat sheet of material can be made to have the illustrated shape resembling the cone end of a trumpet. The slits 15 separating the elements 12 can be made in the flat sheet prior to molding, or can be made to the sheet after it has been molded to its cone end shape.

Thermo-forming of a sheet into the device is very well suited to thermo-plastic materials. Although the sheet that is molded could be a composite made up of a plurality of sheets having differing properties (in the manner of the first and second embodiments), the molding technique is particularly suited to formation of the devices where the sheet-material of the device is not a composite made up of different layers of different materials, but is a single sheet of material having a generally homogenous construction. One such material would be a sheet of condensed polytetrofluoroethylene (cPTFE), such as manufactured by Proxy Biomedical Limited under the trade mark Motifmesh. Motifmesh material combines the favourable ingrowth and healed wound strength characteristics of large pore monofilament polypropylene meshes with the biocompatibility and reduced adhesion attributes of expanded PTFE patches. Motifmesh material has an open pore structure, with an average pore size of 2400 μm and a thickness of 0.15 mm. Further details of Motifmesh material can be found in Proxy Biomedical Limited's WO 2004/006808, the contents of which are hereby incorporated by way of reference.

Although manufacturing the device from a single sheet of material, such as cPTFE Motifmesh material, is particularly suited to a device having the general device configuration illustrated in FIG. 11 and manufactured by the described molding technique, it may also be used for the first and second embodiments. In other words, a device having the general geometry of the first and second embodiments need not be manufactured from a plurality of separate components and need not be manufactured from material comprising a plurality of layers of different materials attached to one another.

Although the device has been described in conjunction with the prevention or treatment of parastomal hernias in patients having a colostomy or ileostomy or the like, the device may also find use in the repair of developed hernias at the site of the passage of other portions of the alimentary canal through the confines of a patient's abdominal cavity. For example, by fitting the collar portion of the device around a patient's oesophagus (with the collar extending downwards towards the patient's stomach) and fastening the generally planar elements to the underside of a patient's diaphragm, the device may be used to repair a developed hiatus hernia in much the same manner that it can be used to repair a developed parastomal hernia. When used for the repair of a developed hiatus hernia it is envisaged that the device will be smaller than when used for the prevention or repair of a parastomal hernia, for example with the diameter of the collar portion reduced in size and the longitudinal extent of the collar portion reduced in length.

1-49. (canceled)

50. A device for use in reinforcing a boundary wall of a patient's abdominal cavity at a site at which a portion of the patient's alimentary tract passes through that boundary wall, the device comprising:
a collar portion provided with a gap along its full length and suitable for extending, in use, around the alimentary tract at said site; and
a plurality of petal-like elements extending from the collar portion, said elements being arranged so as to extend, in use, outwardly with respect to the longitudinal axis of the portion of the alimentary tract passing through the collar portion and being suitable for attachment, in use, to the boundary wall around said site.

51. A device as claimed in claim 50, wherein said petal-like elements have proximal portions, closest to said collar portion, and distal portions, furthest from said collar portion, and at least said distal portions of said petal-like elements are arranged so as to extend, in use, generally radially outwardly with respect to said longitudinal axis.

52. A device as claimed in claim 51, wherein said petal-like elements are generally planar.

53. A device as claimed in claim 52, wherein said petal-like elements are flat.

54. A device as claimed in claim 52, wherein said petal-like elements are, in use, curved.

55. A device as claimed in claim 50, wherein said collar portion is arranged, in use, to be generally tubular, with the longitudinal axis of the portion of the alimentary tract passing through the collar portion being generally co-axial with the longitudinal axis of the tube of the generally tubular collar portion.

56. A device as claimed in claim 50, wherein the collar portion is arranged to extend, in use, only partially around the circumference of the portion of alimentary tract passing through the collar portion, with the alimentary tract's mesentry extending through the gap provided in the collar portion.

57. A device as claimed in claim 56, wherein the collar portion is arranged to extend, in use, around approximately 85%-98% of the circumference of the portion of the alimentary tract passing therethrough.

58. A device as claimed in claim 50, wherein the collar portion is generally cylindrical and is open at its opposite ends.

59. A device as claimed in claim 50, wherein the plurality of petal-like elements are, at least prior to their attachment to the boundary wall, all generally coplanar.

60. A device as claimed in claim 59, wherein the plurality of petal-like elements are sufficiently flexible as to be conformed in use to and into contact with the interior of the boundary wall at said site.

61. A device as claimed in claim 50, wherein, when attached to the interior of the boundary wall around the site, the plurality of petal-like planar elements have a slightly concave shape similar to that of the slightly concave interior shape of the boundary wall around the site.

62. A device as claimed in claim 50, wherein each petal-like element has a pair of lateral edges.

63. A device as claimed in claim 62, wherein the facing lateral edges of at least some of the petal-like elements are generally parallel to one another so that, in use, a plurality of generally triangular areas of the interior of the boundary wall will be left exposed between opposed lateral edges of adjacent petal-like elements.

64. A device as claimed in claim 62, wherein the two lateral edges of at least some of the petal-like elements are not generally parallel to one another.

65. A device as claimed in claim 64, wherein said at least some petal-like elements are generally trapezoidal in shape, so as to reduce the area of the boundary wall interior around the site of the stoma that is left uncovered by the device in use.

66. A device as claimed in claim 65, wherein, in use, the lateral edges of some of the petal-like elements overlap the lateral edges of their immediately neighbouring petal-like elements, thereby to reduce the area of the boundary wall interior left uncovered by the device in use.

67. A device as claimed in claim 50, wherein the material of the collar portion is sufficiently flexible as to enable a surgeon to conform the collar portion of a single device around a variety of different alimentary tracts of different girths.

68. A device as claimed in claim 50, wherein, when free standing prior to use, the device generally resembles an open-topped top hat, with a plurality of generally radially directed slits in the brim of the hat and a single generally radially directed slit through the complete hat.

69. A device as claimed in claim 50, wherein, when freestanding prior to use, the device generally resembles the end of a trumpet, with a plurality of generally radially directed slits in the bell of the trumpet end separating the petal-like elements from one another.

70. A device as claimed in claim 69, wherein the device is flat-packed for storage prior to use, and is required to be manipulated into said open-topped top hat shape or trumpet shape prior to use.

71. A device as claimed in claim 50, wherein the generally petal-like elements are arranged, in use, to be attached to the contacting interior of the boundary wall around the site using sutures, tacks or staples.

72. A device as claimed in claim 50, wherein at least said plurality of petal-like elements comprise a structural biocompatible material.

73. A device as claimed in claim 72, wherein said structural biocompatible material comprises a non-absorbable material and is selected from the group consisting of: polypropylene, dacron, silicon, polyethylene, polyamide, titanium, stainless steel, polymethylmethacrylate, polyurethane and condensed polytetrafluoroethylene (cPTFE).

74. A device as claimed in claim 72, wherein the structural biocompatible material comprises polypropylene mesh or Dacron (polyester) or similar.

75. A device as claimed in claim 72, wherein said plurality of petal-like elements each have a visceral surface, that will face the patient's internal organs in use, and a non-visceral surface that will face the interior of the boundary wall around the site, the visceral surface of said elements comprising a material which will minimise visceral adhesion.

76. A device as claimed in claim 75, wherein the non-visceral surfaces of said elements compose a material which will encourage tissue ingrowth from the non-visceral surface.

77. A device as claimed in claim 76, wherein the non-visceral surfaces of said elements comprise exposed mesh or polypropylene or the like.

78. A device as claimed in claim 77, wherein the material which will minimise tissue attachment comprises sheet-like material, which sheet-like material is attached to the mesh.

79. A device as claimed in claim 78, wherein the sheet-like material extends beyond the extremities of the mesh so as to prevent the patient's internal organs from coming into contact with edges of the mesh in use.

80. A device as claimed in claim 72, wherein the structural biocompatible material comprises a sheet of condensed polytetrofluoroethylene (cPTFE) having an open pore structure.

81. A device as claimed in claim 72, wherein said collar portion has a visceral surface that will face the patient's internal organs in use, and a nonvisceral surface that will face the portion of alimentary tract passing therethrough, the visceral surface of said collar portion comprising a material which will minimise visceral adhesion.

82. A device as claimed in claim 81, wherein the non-visceral surface of said collar portion also comprises a material which will minimise tissue ingrowth from the portion of alimentary tract passing therethrough.

83. A device as claimed in claim 75, wherein said material that will minimise tissue adhesion or ingrowth comprises polytetrafluoroethylene or expanded polytetrafluoroethylene or similar.

84. A device as claimed in claim 50, wherein the collar portion and plurality of petal-like elements are formed from the same sheet of material.

85. A device as claimed in claim 84, wherein the sheet of material is a sheet of condensed polytetrafluoroethylene having an open pore structure.

86. A device as claimed in claim 84, wherein the collar portion and plurality of petal-like elements are molded into shape.

87. A device as claimed in claim 86, wherein the molding involves thermo-forming a single said sheet of material.

88. A device as claimed in claim 50, wherein said boundary wall is the patient's abdominal wall, said portion of said alimentary tract is the patient's intestine and said site is the site of a stoma.

89. A device as claimed in claim 50, wherein said boundary wall is the patient's diaphragm and said portion of said alimentary tract is the patient's oesophagus.

90. A method of reinforcing a boundary wall at a patient's abdominal cavity at a site at which a portion of the patient's alimentary tract passes through that boundary wall, the method comprising:
providing the device of claim 50;
forming the collar portion around the alimentary tract at said site, with said plurality of petal-like elements in contact with the boundary wall around said site; and
fastening said plurality of petal-like elements to the boundary wall around said site.

91. A method as claimed in claim 90, wherein the step of forming the collar portion around the alimentary tract includes slipping the collar portion around the portion of the patient's alimentary canal by passing said alimentary canal portion through the gap and then closing the collar portion around said alimentary canal portion.

92. A method as claimed in claim 90, wherein said boundary wall is the patient's abdominal wall, said portion of said alimentary tract is the patient's intestine and said site is the site of a stoma.

93. A method as claimed in claim 92, wherein, even after closing the collar portion around the intestine, the gap remains, albeit reduced in size, with the intestine's mesentry extending therethrough.

94. A method as claimed in claim 92, wherein the device is a prophylactic device for preventing the subsequent formation of a parastomal hernia and is implanted in the same single surgical procedure as formation of the stoma itself.

95. A method as claimed in claim 90, wherein said boundary wall is the patient's diaphragm and said portion of said alimentary tract is the patient's oesophagus.

96. A method of manufacturing the device of claim 50, comprising:
a) providing a sheet of biocompatible plastics material; and
b) forming said sheet into the collar portion and the plurality of petal-like elements by molding and cutting.

97. A method as claimed in claim 96, wherein the cutting operation is performed on the sheet prior to molding, cuts formed by said cutting operation separating said petal-like elements from one another.

98. A method as claimed in claim 96, wherein the cutting operation is performed on the sheet after molding, cuts formed by said cutting operation separating said petal-like elements from one another.
